(19) United States
(12) Patent Application Publication
(10) Pub. No.: US 2025/0210298 A1
(43) Pub. Date: Jun. 26, 2025

(54) ION SOURCE AND NEUTRON CAPTURE THERAPY APPARATUS

(71) Applicant: SUMITOMO HEAVY INDUSTRIES, LTD., Tokyo (JP)

(72) Inventor: Ryu MURASE, Yokosuka-shi (JP)

(21) Appl. No.: 18/979,030

(22) Filed: Dec. 12, 2024

(30) Foreign Application Priority Data
Dec. 22, 2023 (JP) 2023-216639

(51) Int. Cl. H01J 37/08; H01J 37/32

(52) U.S. Cl. CPC H01J 37/08; H01J 37/32541; H01J 37/3255

(57) ABSTRACT

An ion source includes a plasma electrode that is provided in a plasma chamber in which ions are generated by a plasma; and an extraction electrode that faces the plasma electrode and extracts the ions from the plasma chamber. The plasma electrode includes at least a first electrode member and a second electrode member, and is formed with an extraction opening. The first electrode member is a magnetic body. The second electrode member is a member having at least one of a melting point and thermal conductivity higher than those of the magnetic body. In a second direction perpendicular to a first direction, a distance of an outer peripheral side end portion of the first electrode member from the extraction opening is equal to or greater than a distance of an outer peripheral side end portion of the extraction electrode from the extraction opening.

### CROSS-REFERENCE TO RELATED APPLICATIONS

This application claims priority to Japanese Patent Application No. 2023-216639, filed on Dec. 22, 2023, which is incorporated by reference herein in its entirety.

### BACKGROUND

### Technical Field

A certain embodiment of the present invention relates to an ion source and a neutron capture therapy apparatus.

### Description of Related Art

There is known an ion source configured to generate plasma in a plasma chamber and extract ions from the plasma chamber using an extraction electrode (for example, refer to the related art). The ions generated in the plasma chamber are extracted to the outside of the plasma chamber by a potential difference between a plasma electrode and an extraction electrode of the plasma chamber.

### SUMMARY

According to an embodiment of the present invention, there is provided an ion source including a plasma electrode that is provided in a plasma chamber in which ions are generated by a plasma; and an extraction electrode that faces the plasma electrode and extracts the ions from the plasma chamber. The plasma electrode includes at least a first electrode member and a second electrode member, and is formed with an extraction opening through which the ions are extracted. The first electrode member is a magnetic body. The second electrode member is a member having at least one of a melting point and thermal conductivity higher than those of the magnetic body. In a second direction perpendicular to a first direction in which the plasma electrode and the extraction electrode face each other, a distance of an outer peripheral side end portion of the first electrode member from the extraction opening is equal to or greater than a distance of an outer peripheral side end portion of the extraction electrode from the extraction opening.

According to another embodiment of the present invention, there is provided a neutron capture therapy apparatus including an accelerator that includes an ion source for generating ions, and that accelerates the ions to emit a particle ray; and an irradiation unit that generates a neutron ray by the particle ray and irradiates an object with the neutron ray. The ion source includes a plasma electrode that is provided in a plasma chamber in which ions are generated by a plasma, and an extraction electrode that faces the plasma electrode and extracts the ions from the plasma chamber. The plasma electrode includes at least a first electrode member and a second electrode member, and is formed with an extraction opening through which the ions are extracted. The first electrode member is a magnetic body. The second electrode member is a member having at least one of a melting point and thermal conductivity higher than those of the magnetic body. In a second direction perpendicular to a first direction in which the plasma electrode and the extraction electrode face each other, a distance of an outer peripheral side end portion of the first electrode member from the extraction opening is equal to or greater than a distance of an outer peripheral side end portion of the extraction electrode from the extraction opening.

### BRIEF DESCRIPTION OF THE DRAWINGS

FIG. 1 is a schematic view illustrating a neutron capture therapy apparatus 100 including an ion source 10 according to an embodiment of the present invention.

FIG. 2 is a schematic configuration diagram of the ion source 10.

FIG. 3 is a schematic configuration diagram in which the vicinity of a plasma chamber of the ion source 10 is enlarged.

FIG. 4 is a sectional view illustrating the configuration of a plasma electrode 90 illustrated in FIG. 3 in more detail.

FIG. 5 illustrates a state where an outer peripheral side end portion 91b of a first electrode member 91 is disposed at the same position (on a reference line SL1) as an outer peripheral side end portion 44b of the first extraction electrode 44 in a radial direction.

FIG. 6 illustrates a state where the outer peripheral side end portion 91b of the first electrode member 91 is disposed at the same position (on a reference line SL2) as an inner peripheral side end portion 84a of a coil 84 in the radial direction.

FIG. 7 is a diagram illustrating results obtained by creating a model of the ion source 10 and simulating a two-dimensional magnetic field calculation system.

FIG. 8 is a diagram illustrating models of Example 1 to Example 5.

FIG. 9 is a diagram illustrating simulation results of the magnetic field intensity of Example 1 to Example 5.

FIGS. 10A and 10B are diagrams illustrating models of Example 5 and Example 6.

FIG. 11 is a diagram illustrating simulation results of the magnetic field intensity of Example 5 and Example 6.

FIG. 12 is a schematic sectional view illustrating a second electrode member of an ion source according to a modification example.

### DETAILED DESCRIPTION

For example, in a case where plasma is generated by inputting microwaves, when a magnetic field coaxial with a direction in which the ions are extracted is formed by a coil or the like, the microwave absorption efficiency is improved, and the efficiency of generating the ions is improved. However, a strong electric field is applied between the extraction electrode and the plasma electrode, and when a magnetic field leaks to this point, the following problem occurs. That is, electrons move so as to be wrapped around the magnetic field. Discharge occurs between the electrodes due to such electrons. In a case where the discharge occurs, there is a case where the plasma electrode is damaged.

Therefore, it is desirable to provide an ion source and a neutron capture therapy apparatus capable of reducing the influence of discharge between electrodes.

In the ion source according to the present invention, the plasma electrode includes at least the first electrode member and the second electrode member, and the first electrode member is a magnetic body. Accordingly, a magnetic field is allowed to escape to the outer peripheral side by the first electrode member which is a magnetic body, so that a leaked magnetic field to between the plasma electrode and the extraction electrode can be suppressed. In particular, in the second direction perpendicular to the first direction in which the plasma electrode and the extraction electrode face each other, the distance from the outer peripheral side end portion of the first electrode member to the extraction opening is equal to or greater than the distance from the outer peripheral side end portion of the extraction electrode to the extraction opening. Accordingly, it is possible to suppress the generation of a leaked magnetic field from the outer peripheral side of the first electrode member. In this way, by suppressing the generation of a leaked magnetic field, the generation of the discharge between the plasma electrode and the extraction electrode can be suppressed. In addition, the second electrode member is a member having at least one of a melting point and thermal conductivity higher than those of the magnetic body. Therefore, even when the discharge occurs, the first electrode member, which is the magnetic body, can be protected by the second electrode member. From the above, the influence of the discharge between the electrodes can be reduced. In addition, by suppressing the damage to the first electrode member, the frequency of failure and the frequency of maintenance can be reduced.

The material of the second electrode member may be selected from tungsten, tantalum, and molybdenum. In this case, the second electrode member can be made to have a high melting point.

The material of the second electrode member may be selected from copper, silver, and aluminum. In this case, the second electrode member can be made to have high thermal conductivity.

The ion source may further include a magnetic field generation unit that is provided on an outer peripheral side of the plasma chamber and generates a magnetic field in the first direction in the plasma chamber. The outer peripheral side end portion of the first electrode member may be disposed at a position separated from the magnetic field generation unit toward an inner peripheral side. Even in this case, it is possible to suppress the generation of a leaked magnetic field from the outer peripheral side end portion of the first electrode member.

The ion source may further include a magnetic field generation unit that is provided on an outer peripheral side of the plasma chamber and generates a magnetic field in the first direction in the plasma chamber. The first electrode member may reach the magnetic field generation unit. In this case, it is possible to further suppress the generation of a leaked magnetic field from the outer peripheral side end portion of the first electrode member.

The magnetic field generation unit may include a coil and a yoke, and the first electrode member may include a connecting part that is connected to the yoke of the magnetic field generation unit. In this case, the connecting part allows the first electrode member to be connected to a magnetic circuit with the coil, and the generation of a leaked magnetic field can be further suppressed.

In the neutron capture therapy apparatus according to the present invention, the plasma electrode has at least the first electrode member and the second electrode member, and the first electrode member is a magnetic body. Accordingly, a magnetic field is allowed to escape to the outer peripheral side by the first electrode member which is a magnetic body, so that a leaked magnetic field to between the plasma electrode and the extraction electrode can be suppressed. In particular, in the second direction perpendicular to the first direction in which the plasma electrode and the extraction electrode face each other, the distance from the outer peripheral side end portion of the first electrode member to the extraction opening is equal to or greater than the distance from the outer peripheral side end portion of the extraction electrode to the extraction opening. Accordingly, it is possible to suppress the generation of a leaked magnetic field from the outer peripheral side of the first electrode member. In this way, by suppressing the generation of a leaked magnetic field, the generation of the discharge between the plasma electrode and the extraction electrode can be suppressed. In addition, the second electrode member is a member having at least one of a melting point and thermal conductivity higher than those of the magnetic body. Therefore, even when the discharge occurs, the first electrode member, which is the magnetic body, can be protected by the second electrode member. From the above, the influence of the discharge between the electrodes can be reduced. In addition, by suppressing the damage to the first electrode member, it is possible to reduce the frequency of a failure that causes the neutron ray to be unable to be emitted, and it is possible to reduce the frequency of maintenance.

Hereinafter, a preferred embodiment of the present invention will be described in detail with reference to the drawings.

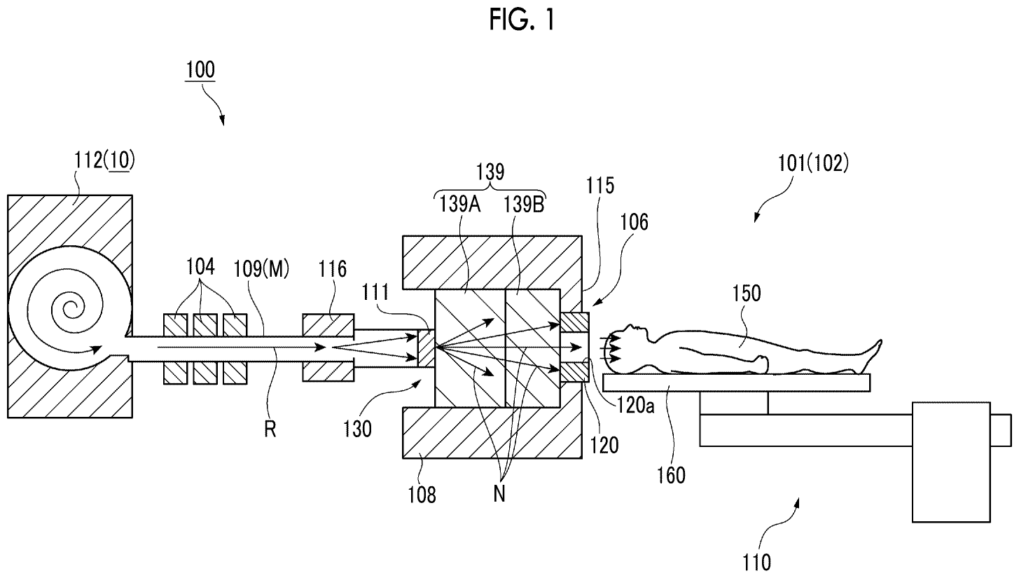

FIG. 1 is a schematic view illustrating a neutron capture therapy apparatus 100 including an ion source 10 according to an embodiment of the present invention. The neutron capture therapy apparatus 100 is an apparatus that performs cancer treatment using a boron neutron capture therapy (BNCT). The neutron capture therapy apparatus 100 includes a treatment unit 102, a treatment bed 160, and a moving mechanism 110.

The treatment unit 102 (irradiation unit) has an irradiation port 106 for irradiating a patient 150 with a neutron ray N. The treatment unit 102 is configured by a structure in which the irradiation port 106 and the moving mechanism 110 are disposed. The treatment unit 102 is provided in the treatment room 101. The irradiation port 106 is provided on a vertical wall portion of the treatment room 101. The neutron ray N is emitted from the irradiation port 106 in a horizontal direction. The irradiation port 106 includes a collimator 120 (to be described below) and an irradiation unit peripheral wall 115. The moving mechanism 110 is a mechanism that is capable of moving the treatment bed 160 on which the patient 150 is placed in the treatment unit 102. The moving mechanism 110 is provided at a position in front of the irradiation port 106 in the treatment room 101.

In the treatment unit 102, for example, the tumor of the patient 150 to whom boron (10B) is administered is irradiated with the neutron ray N. The irradiation port 106 irradiates the patient 150 placed on the treatment bed 160 with the neutron ray N (particle ray).

The neutron capture therapy apparatus 100 includes an accelerator 112. The accelerator 112 has an ion source 10 (to be described below). The accelerator 112 accelerates the ions generated by the ion source to emit the particle ray R. For example, a cyclotron, a linear accelerator, or the like may be adopted as the accelerator 112.

The particle ray R emitted from the accelerator 112 passes through a transport path 109, which is internally maintained in vacuum and internally allows a beam to pass therethrough and is referred to as a beam duct, and is transported to a target disposition section 130. The target disposition section 130 is a portion where a target 111 is disposed, and has a mechanism that holds the target 111 in a posture when the target 111 is irradiated. The target disposition section 130 disposes the target 111 at a position facing an end portion (emission port) of the transport path 109. The particle ray R emitted from the accelerator 112 passes through the transport path 109 and travels toward the target 111 disposed at the end portion of the transport path 109. A plurality of electromagnets 104 (quadrupole electromagnets or the like) and a scanning electromagnet 116 are provided along the transport path 109. The plurality of electromagnets 104 are, for example, electromagnets that perform beam axis adjustment of the particle ray R using the electromagnets.

The scanning electromagnet 116 scans the particle ray R and controls the irradiation of the target 111 with the particle ray R. The scanning electromagnet 116 controls an irradiation position of the particle ray R with respect to the target 111.

In addition, the neutron capture therapy apparatus 100 generates the neutron ray N by irradiating the target 111 with the particle ray R and emits the neutron ray N toward the patient 150. The neutron capture therapy apparatus 100 includes the target 111, a shield member 108, a deceleration member 139, and a collimator 120.

When the target 111 is irradiated with the particle ray R, the neutron ray N is generated. The target 111 is a solid-shaped member formed of a material that generates the neutron ray N by being irradiated with the particle ray R. Specifically, the target 111 is formed of, for example, beryllium (Be), lithium (Li), tantalum (Ta), or tungsten (W), and has, for example, a disk-shaped solid shape having a diameter of 160 mm. The shape of the target 111 is not limited to the disk shape and may be another shape.

The deceleration member 139 decelerates the neutron ray N generated by the target 111 (reduces the energy of the neutron ray N). The deceleration member 139 may have a laminated structure including a layer 139A that mainly decelerates the fast neutrons included in the neutron ray N and a layer 139B that mainly decelerates the epithermal neutrons included in the neutron ray N.

The shield member 108 blocks the generated neutron ray N and a gamma ray or the like generated due to the generation of the neutron ray N such that the rays are not released to the outside. The shield member 108 is provided to surround the deceleration member 139. The upper and lower portions of the shield member 108 extend to the upstream side of the particle ray R from the deceleration member 139.

The collimator 120 shapes the irradiation field of the neutron ray N and includes an irradiation port 120a through which the neutron ray N passes. The collimator 120 is, for example, a block-shaped member having the irradiation port 120a at the center. The collimator 120 is attached to the irradiation unit peripheral wall 115, which is a wall portion of the point where the inside of the treatment room 101 is irradiated with the neutron ray N.

The moving mechanism 110 is a so-called 6-axis mechanism that moves the treatment bed 160 on which the patient 150 is placed in 6-axis directions. The moving mechanism 110 enables the horizontal movement and rotational movement of the treatment bed 160. In the present embodiment, the moving mechanism 110 supports the patient 150 placed on the treatment bed 160, and moves the patient 150 together with the treatment bed 160.

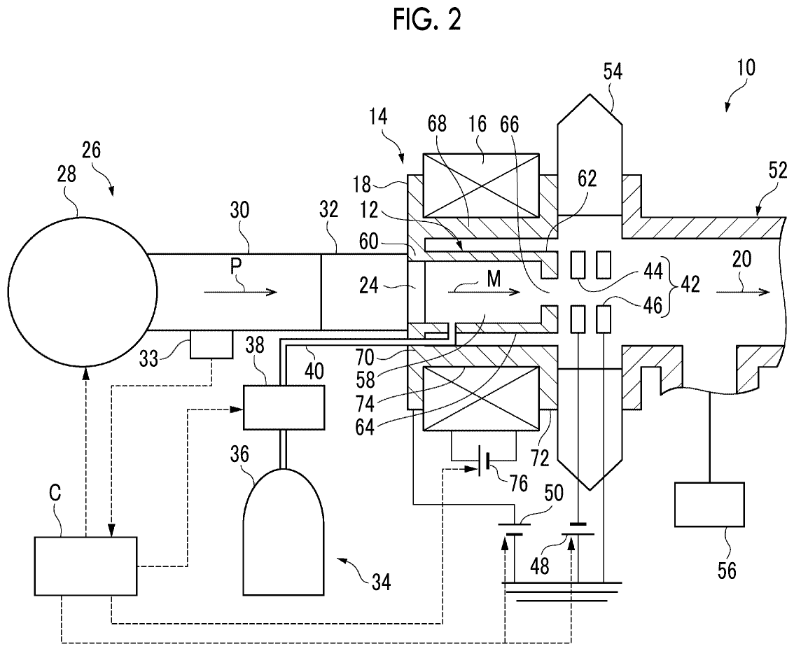

Next, a detailed configuration of the ion source 10 will be described with reference to FIG. 2. FIG. 2 is a schematic configuration diagram of the ion source 10. As illustrated in FIG. 2, the ion source 10 is an ion source that generates a high-density plasma and extracts ions by inputting microwave power in a magnetic field direction into a plasma chamber 12 to which a magnetic field satisfying an electron cyclotron resonance (ECR) condition or a magnetic field higher than that of the magnetic field satisfying the ECR condition is applied. The ion source 10 is configured to generate a plasma of a raw material gas by an interaction between a magnetic field and a microwave and to extract ions from the plasma to the outside of the plasma chamber 12.

The intensity of the magnetic field satisfying the ECR condition is uniquely determined with respect to the frequency of the microwave used, and a magnetic field of 87.5 mT (875 gauss) is required in a case where the microwave frequency is, for example, 2.45 GHz. Hereinafter, for convenience of description, a magnetic field satisfying the ECR condition may be referred to as a resonance magnetic field.

The ion source 10 includes an ion source main body 14. The ion source main body 14 includes the plasma chamber 12, a magnetic field generator 16 (magnetic field generation unit), and a vacuum chamber 18.

The plasma chamber 12 has a cylindrical shape with both ends. In the following, a direction from one end of the plasma chamber 12 to the other end will be referred to as an axial direction for convenience. In addition, a direction perpendicular to the axial direction is referred to as the radial direction, and a direction surrounding the axial direction is referred to as a circumferential direction. However, these do not necessarily mean that the plasma chamber 12 has a shape with rotational symmetry. The axial direction corresponds to a “first direction” in the claims. The radial direction corresponds to a “second direction” in the claims. In the illustrated example, the plasma chamber 12 has a cylindrical shape. However, the plasma chamber 12 may have any shape as long as the plasma chamber 12 can appropriately accommodate the plasma. In addition, the axial length of the plasma chamber 12 may be longer than or shorter than the radial length of an end portion of the plasma chamber 12.

The magnetic field generator 16 is provided to apply a magnetic field to the plasma chamber 12. The magnetic field generator 16 is disposed around the plasma chamber 12. The magnetic field generator 16 is configured to generate a magnetic field along a center axis of the plasma chamber 12. The magnetic field direction is indicated by an arrow M in FIG. 2. The magnetic field generator 16 is configured to generate a resonance magnetic field or a magnetic field having a higher intensity than the resonance magnetic field in at least a portion on the axis of the plasma chamber 12. The magnetic field generator 16 can also generate a magnetic field lower than the resonance magnetic field in at least a portion on the axis of the plasma chamber 12.

The vacuum chamber 18 is a casing for accommodating the plasma chamber 12 in a vacuum environment. The vacuum chamber 18 is also a structure for holding the magnetic field generator 16. The plasma chamber 12 has a vacuum window 24 for receiving a microwave therein. The plasma chamber 12, the magnetic field generator 16, and the vacuum chamber 18 will be described in more detail below.

The ion source 10 includes a microwave supply system 26. The microwave supply system 26 is configured to input microwave power into the plasma chamber 12 through the vacuum window 24. The microwave supply system 26 includes a microwave source 28, a waveguide 30, and a matching section 32. The microwave source 28 is, for example, a magnetron. The microwave source 28 outputs, for example, a microwave having a frequency of 2.45 GHz. The waveguide 30 is a three-dimensional circuit for transmitting the microwave output by the microwave source 28 to the plasma chamber 12. One end of the waveguide 30 is connected to the microwave source 28, and the other end thereof is connected to the vacuum window 24 via the matching section 32. The matching section 32 is provided for matching of the microwave.

In this way, the microwave is introduced into the plasma chamber 12 from the microwave supply system 26 through the vacuum window 24. The introduced microwave propagates to the inside of the plasma chamber 12 toward the end portion of the plasma chamber 12 facing the vacuum window 24. The propagation direction of the microwave is indicated by an arrow P in FIG. 2. The propagation direction P of the microwave is the same direction as the magnetic field direction M by the magnetic field generator 16. Thus, the propagation direction P of the microwave coincides with the axial direction of the plasma chamber 12.

In addition, the microwave supply system 26 includes a microwave detector 33 provided in the waveguide 30. The microwave detector 33 includes, for example, a directional coupler for monitoring incident power to the plasma chamber 12 and reflected power from the plasma chamber 12. The microwave detector 33 is configured to output a measurement result to a control device C.

The ion source 10 includes a gas supply system 34. The gas supply system 34 is configured to supply the raw material gas for the plasma to the plasma chamber 12. The gas supply system 34 includes a gas cylinder 36 serving as a gas source and a gas flow rate controller 38. A tip of a gas pipe 40 of the gas supply system 34 is connected to the plasma chamber 12 through the vacuum chamber 18. For example, the gas pipe 40 is connected to a side wall 64 of the plasma chamber 12. The gas flow rate controller 38 includes an on-off valve for connecting or blocking the gas cylinder 36 to or from the plasma chamber 12, or a flow rate control valve for adjusting the gas flow rate from the gas cylinder 36 to the plasma chamber 12. In this way, the raw material gas is supplied to the plasma chamber 12 at a controlled flow rate from the gas cylinder 36.

The ion source main body 14 includes an extraction electrode system 42. The extraction electrode system 42 is configured to extract ions from the plasma through an extraction opening 66 of the plasma chamber 12. The extraction electrode system 42 includes a first extraction electrode 44 and a second extraction electrode 46. The first extraction electrode 44 is provided between the plasma chamber 12 and the second extraction electrode 46. A trailing end portion 62 having the extraction opening 66 and the first extraction electrode 44 are arrayed with a gap therebetween, and the first extraction electrode 44 and the second extraction electrode 46 are arrayed with a gap therebetween. The first extraction electrode 44 and the second extraction electrode 46 are formed in an annular shape, for example, respectively, and have an opening portion for passing the ions extracted from the plasma chamber 12 at a center portion thereof.

The first extraction electrode 44 is provided to extract positive ions from the plasma and to prevent the electrons from returning from a beamline 52 to the plasma chamber 12. For that purpose, a negative high voltage is applied to the first extraction electrode 44. A first extraction power supply 48 is provided in order to apply a negative high voltage to the first extraction electrode 44. The second extraction electrode 46 is grounded. In addition, a positive high voltage is applied to the vacuum chamber 18. A second extraction power supply 50 is provided in order to apply a positive high voltage to the vacuum chamber 18. The absolute value of the positive high voltage applied to the vacuum chamber 18 is larger than the absolute value of the negative high voltage applied to the first extraction electrode 44. In this way, an ion beam 20 of the positive ions is extracted from the plasma chamber 12. The extraction direction of the ion beam 20 from the plasma chamber 12 is the same direction as the propagation direction P of the microwave.

The ion source 10 is provided with the beamline 52 for transporting the ion beam 20 extracted by the extraction electrode system 42. The beamline 52 is connected to the ion source main body 14 on a side opposite to the microwave supply system 26. The beamline 52 is a vacuum chamber that communicates with the vacuum chamber 18. The beamline 52 is insulated from the vacuum chamber 18 of the ion source main body 14 and is attached to the vacuum chamber 18. For that purpose, a bushing 54 is provided between the beamline 52 and the vacuum chamber 18.

The bushing 54 maintains the vacuum of the beamline 52 and the vacuum chamber 18 and holds a withstand voltage between the vacuum chamber 18 and the ground side. The bushing 54 is formed of an insulating material. The bushing 54 has an annular shape, and surrounds the extraction electrode system 42. The bushing 54 is interposed and attached between attachment flanges of the respective vacuum chambers of the beamline 52 and the ion source main body 14.

An evacuation system 56 for providing a vacuum environment to the vacuum chamber 18 and the plasma chamber 12 is provided. In the illustrated example, the evacuation system 56 is provided in the beamline 52. Since the beamline 52 communicates with the vacuum chamber 18 and the plasma chamber 12, the evacuation system 56 can evacuate the vacuum chamber 18 and the plasma chamber 12. The evacuation system 56 includes, for example, a high vacuum pump such as a cryopump or a turbo molecular pump.

The ion source 10 may include the control device C for controlling the output of the ion beam 20. The control device C controls each component of the ion source 10, controls the plasma generated in the plasma chamber 12, and thereby controls the output of the ion beam 20. For example, the control device C is configured to control the operation of the microwave supply system 26, the gas supply system 34, and a coil power supply 76. For example, the control device C may control the output of the ion beam 20 by adjusting at least one of the flow rate of the raw material gas, the microwave power, and the magnetic field intensity.

The plasma chamber 12 is configured to generate and maintain the plasma in an internal space thereof. Hereinafter, an internal space of the plasma chamber 12 may be referred to as a plasma generation space 58.

The plasma chamber 12 includes a leading end portion 60, the trailing end portion 62, and the side wall 64. The leading end portion 60 and the trailing end portion 62 face each other with the plasma generation space 58 interposed therebetween. The side wall 64 surrounds the plasma generation space 58 and connects the leading end portion 60 and the trailing end portion 62. In this way, the plasma generation space 58 is defined inside the vacuum chamber 18 by the leading end portion 60, the trailing end portion 62, and the side wall 64. In a case where the plasma chamber 12 has a cylindrical shape, the leading end portion 60 and the trailing end portion 62 have a disk shape, the side wall 64 has a cylindrical shape, and terminals of the side wall 64 are fixed to outer peripheral portions of the leading end portion 60 and the trailing end portion 62.

The leading end portion 60 has the vacuum window 24. The vacuum window 24 may occupy the entire leading end portion 60, or may be formed in a part (for example, a center portion) of the leading end portion 60. One side of the vacuum window 24 faces the plasma generation space 58, and the other side of the vacuum window 24 is directed toward the microwave supply system 26. The vacuum window 24 seals the inside of the plasma chamber 12 in vacuum. The propagation direction P of the microwave is vertical to the vacuum window 24. The vacuum window 24 is formed of a dielectric having low dielectric loss (for example, alumina, boron nitride, or the like). In addition, a portion of the plasma chamber 12 other than the vacuum window 24 is formed of a non-magnetic metallic material such as stainless steel or aluminum.

At least one extraction opening 66 is formed in the trailing end portion 62. The extraction opening 66 is formed at a position facing the vacuum window 24 with the plasma generation space 58 interposed therebetween. That is, the vacuum window 24, the plasma generation space 58, and the extraction opening 66 are arrayed in the axial direction of the plasma chamber 12.

The vacuum chamber 18 has a double cylinder structure in which the plasma chamber 12 is integrally formed. That is, the plasma chamber 12 is an inner cylinder of the vacuum chamber 18, and an outer cylinder 68 that accommodates the plasma chamber 12 is provided outside the plasma chamber 12. The outer cylinder 68 may have a cylindrical shape coaxial with the plasma chamber 12. A gap is provided between the outer cylinder 68 and the side wall 64 of the plasma chamber 12, and a tip part of the gas pipe 40 of the above-described gas supply system 34 enters the gap and is attached to the side wall 64. The vacuum chamber 18 is formed of, for example, a non-magnetic metallic material.

The vacuum chamber 18 may not be formed integrally with the plasma chamber 12.

The vacuum chamber 18 and the plasma chamber 12 may be separate bodies and may be separable from each other. In addition, the vacuum chamber 18 itself may form the plasma chamber 12. In a case where the vacuum chamber 18 is used as the plasma chamber 12 in this way, an end plate having the extraction opening 66 may be attached to the beamline 52 side of the outer cylinder 68.

One end of the vacuum chamber 18 is closed by an end plate 70, and the other end thereof is open toward the beamline 52. The leading end portion 60 of the plasma chamber 12 is formed in a center portion of the end plate 70. An outer peripheral portion of the end plate 70 extends to the outside of the outer cylinder 68 in the radial direction. An attachment flange 72 for the bushing 54 is provided at the end portion of the vacuum chamber 18 on the beamline 52 side. The attachment flange 72 extends outward in the radial direction from the outer cylinder 68. The vacuum chamber 18 and the plasma chamber 12 have the same axial length, and the attachment flange 72 and the trailing end portion 62 of the plasma chamber 12 have the same axial position. The vacuum chamber 18 and the plasma chamber 12 may have different axial lengths.

A magnet holding unit 74 for holding the magnetic field generator 16 is formed in the vacuum chamber 18. The magnet holding unit 74 is formed on an outer surface of the outer cylinder 68 of the vacuum chamber 18, for example. In the present example, the magnetic field generator 16 is provided outside the vacuum chamber 18 (that is, in the atmosphere). The magnetic field generator 16 is disposed to surround the vacuum chamber 18. However, in another example, the vacuum chamber 18 may include a magnet holding unit 74 for holding the magnetic field generator 16 inside the vacuum chamber 18 (that is, in vacuum). In this case as well, the same effects as those in the present example can be obtained. In this way, the magnetic field generator 16 is disposed to surround the plasma generation space 58.

The magnetic field generator 16 includes a coil configured to generate a magnetic field directed in the axial direction of the plasma chamber 12. In the present example, the plasma chamber 12 and the vacuum chamber 18 have a cylindrical shape, the coil is formed in an annularshape, and a wire is wound in the circumferential direction of the plasma chamber 12. The magnetic field generator 16 includes the coil power supply 76 for applying a current to the coil. The number of coils included in the magnetic field generator 16 is not particularly limited, and the magnetic field generator 16 may include one coil or may include a plurality of coils arrayed in the axial direction of the plasma chamber 12.

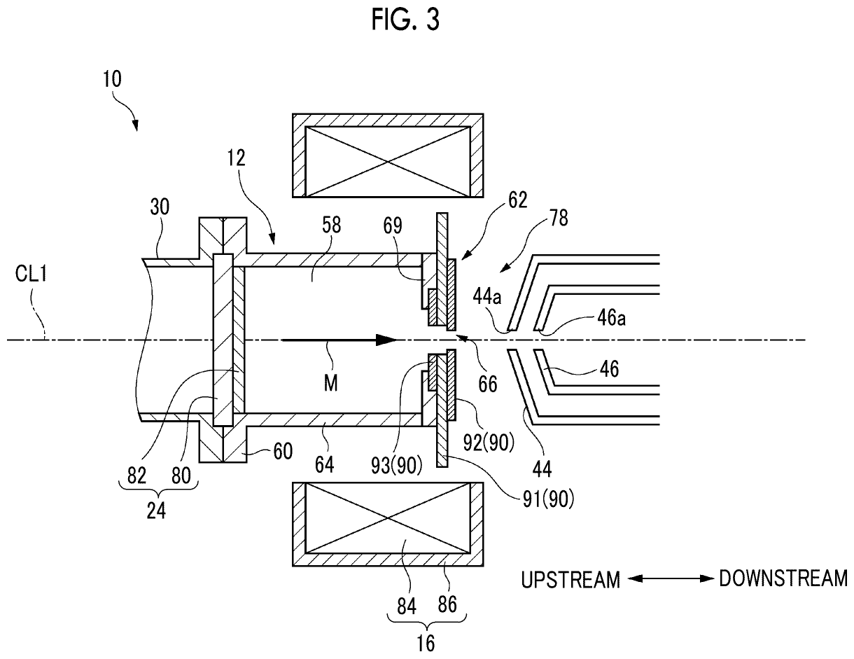

FIG. 3 is a schematic configuration diagram in which the vicinity of the plasma chamber of the ion source 10 is enlarged. As illustrated in FIG. 3, the ion source 10 includes the above-described plasma chamber 12 that generates ions by the plasma, and the extraction electrodes 44 and 46 for extracting ions to the outside from the extraction opening 66 of the plasma chamber 12. The plasma chamber 12 includes the trailing end portion 62 that defines the terminal of the plasma generation space 58 in the axial direction. The trailing end portion 62 faces the first extraction electrode 44 with an extraction gap 78 therebetween in the axial direction. In FIG. 3, a center axis CL1 of the plasma chamber 12 is illustrated. A direction in which the center axis CL1 extends is the axial direction. In addition, in the axial direction, a side from which ions are extracted may be referred to as a “downstream” side, and an opposite side may be referred to as an “upstream” side. In addition, a side far from the center axis CL1 may be referred to as an “outer peripheral” side, and a side close to the center axis CL1 may be referred to as an “inner peripheral” side.

Meanwhile, the vacuum window 24 is provided in the leading end portion 60 of the plasma chamber 12. The vacuum window 24 has a two-layer structure including a window body 80 and a window protection material 82. The window protection material 82 is an inner layer of the vacuum window 24 facing the plasma generation space 58, and the window body 80 is an outer layer of the vacuum window 24 adjacent to the window protection material 82 on the waveguide 30 side. The window protection material 82 covers the window body 80 in order to protect the window body 80 from electrons flowing back to the plasma chamber 12 from the outside of the plasma chamber 12 through the extraction opening 66. The window body 80 is, for example, a plate made of alumina, and the window protection material 82 is, for example, a plate made of boron nitride. In order to protect the side wall 64 of the plasma chamber 12 from the plasma, a liner (for example, made of boron nitride) that covers the inner surface of the side wall 64 may be provided.

The magnetic field generator 16 that generates a magnetic field in the axial direction is provided around the side wall 64 of the plasma chamber 12. The magnetic field generator 16 includes a donut-shaped coil 84 that surrounds the plasma chamber 12 around the center axis CL1, and a yoke 86 mounted on the coil 84. The yoke 86 is provided adjacent to the outer periphery and both axial end portions of the coil 84. As the material of the yoke 86, a magnetic material such as iron may be adopted.

The trailing end portion 62 includes an end wall 69 and a plasma electrode 90. The end wall 69 is a wall that extends from the end portion of the side wall 64 on the downstream side to the inner peripheral side. The plasma electrode 90 is an electrode provided in the plasma chamber 12 and having the extraction opening 66 that is open from the plasma chamber 12 in the axial direction. The plasma electrode 90 spreads vertically to the axial direction toward the outer peripheral side around the center axis CL1. The plasma electrode 90 has an axisymmetric shape with the center axis CL1 as a reference. The above-described extraction opening 66 penetrating in the axial direction is formed in the center of the plasma electrode 90. A high voltage is applied to the plasma electrode 90 (second electrode member 92) by the above-described second extraction power supply 50 (refer to FIG. 2).

The plasma electrode 90 has a first electrode member 91, a second electrode member 92, and a third electrode member 93. The second electrode member 92 is provided on the downstream side of the first electrode member 91 in the axial direction. The third electrode member 93 is provided on the upstream side of the first electrode member 91 in the axial direction.

The first electrode member 91 is a member for suppressing a leaked magnetic field from the plasma chamber 12. The first electrode member 91 is a magnetic body.

Specifically, as the magnetic body, a soft magnetic body such as iron may be adopted. As the magnetic body, cobalt, nickel, or the like may be adopted. The second electrode member 92 is a member for protecting the first electrode member 91 from discharge in a case where the discharge occurs between the first extraction electrode 44 and the plasma electrode 90. The second electrode member 92 is a member having at least one of a melting point and thermal conductivity higher than those of the magnetic body of the first electrode member 91. A non-magnetic body may be adopted as the second electrode member 92. As the material of the second electrode member 92, a material selected from tungsten, tantalum, and molybdenum, which is a high melting point material, may be adopted. As the material of the second electrode member 92, a material having high thermal conductivity may be adopted, and the material may be selected from copper, silver, and aluminum. The third electrode member 93 is a member for protecting the first electrode member 91 from the plasma. As the third electrode member 93, for example, an insulating material having plasma resistance such as boron nitride or alumina may be adopted.

The first extraction electrode 44 faces the plasma electrode 90 in the axial direction at a position separated from the plasma electrode 90 to the downstream side. The second extraction electrode 46 faces the first extraction electrode 44 in the axial direction at a position separated from the first extraction electrode 44 to the downstream side. The extraction electrodes 44 and 46 have openings 44a and 46a through which the extracted ions pass. In the present embodiment, the extraction electrodes 44 and 46 have a conical shape such that the extraction electrodes 44 and 46 face the upstream side in the axial direction from the outer peripheral side to the inner peripheral side thereof. Therefore, the first extraction electrode 44 is closest to the plasma electrode 90 at a position in the vicinity of the opening 44a.

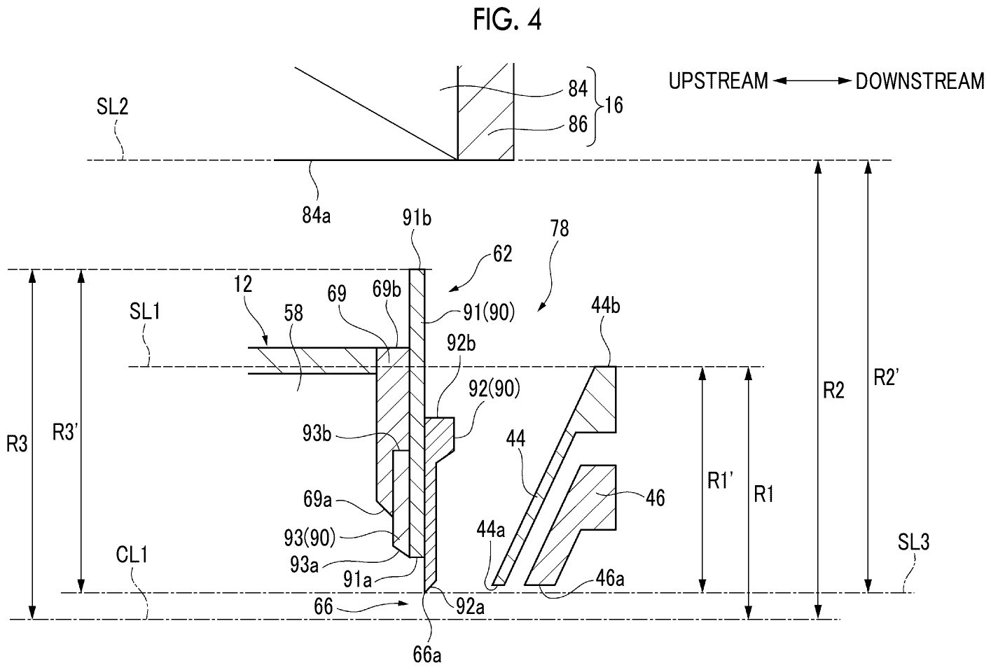

Next, FIG. 4 is a sectional view illustrating the configuration of the plasma electrode 90 illustrated in FIG. 3 in more detail. A positional relationship of the plasma electrode 90 will be described with reference to FIG. 4. A reference line SL1 extending in the axial direction (the first direction) is set with respect to an outer peripheral side end portion 44b of the first extraction electrode 44. The radius (outer diameter) of the outer peripheral side end portion 44b of the first extraction electrode 44, that is, the distance of the end portion 44b in the radial direction (the second direction) from the center axis CL1 is defined as a distance R1. A reference line SL2 extending in the axial direction is set with respect to an inner peripheral side end portion 84a of the coil 84. The radius (inner diameter) of the inner peripheral side end portion 84a of the coil 84, that is, the distance of the inner peripheral side end portion 84a in the radial direction from the center axis CL1 is defined as a distance R2. The distances of the reference lines SL1 and SL2 in the radial direction from the center axis CL1 are constant at each position in the axial direction.

The extraction opening 66 is formed by a through-hole penetrating the end wall 69, the first electrode member 91, the second electrode member 92, and the third electrode member 93 in the axial direction. In the present embodiment, the extraction opening 66 is configured by a circular through-hole having the center axis CL1 as a center. However, the shape of the extraction opening 66 may be any shape as long as ions can be extracted from the plasma chamber 12, and the shape, size, and the like are not particularly limited. As illustrated in FIG. 4, the inner diameters of the extraction opening 66 formed in the end wall 69, the first electrode member 91, the second electrode member 92, and the third electrode member 93 may not be the same as each other, and may have different inner diameters. The extraction opening 66 has an inner peripheral edge 66a. The inner peripheral edge 66a is the point of the extraction opening 66 where the inner diameter is the smallest. In the present embodiment, a tip part of an inner peripheral edge of the second electrode member 92 is the inner peripheral edge 66a. However, an inner peripheral edge of another member may be the inner peripheral edge 66a. A reference line SL3 extending in the axial direction (the first direction) is set with respect to the inner peripheral edge 66a of the extraction opening 66. The radius (outer diameter) of the outer peripheral side end portion 44b of the first extraction electrode 44, that is, the distance of the end portion 44b in the radial direction (the second direction) from the extraction opening 66 (here, the reference line SL3) is defined as a distance R1′. The radius (inner diameter) of the inner peripheral side end portion 84a of the coil 84, that is, the distance of the end portion 84a from the extraction opening 66 (here, the reference line SL3) in the radial direction is defined as a distance R2′. The distances of the reference lines SL1 and SL2 from the reference line SL3 in the radial direction are constant at each position in the axial direction.

The first electrode member 91 of the plasma electrode 90 has a disk shape that spreads in the radial direction around the center axis CL1. The first electrode member 91 has an inner peripheral side end portion 91a and an outer peripheral side end portion 91b. A positional relationship between the inner peripheral side end portion 91a of the first electrode member 91 and the inner peripheral side end portion 69a of the end wall 69 can be appropriately changed within a range in which the extraction of the ions is not affected. However, in the example illustrated in FIG. 4, the inner peripheral side end portion 91a is disposed on the inner peripheral side of the end wall 69 and is disposed on the outer peripheral side of the inner peripheral side openings 44a and 46a of the extraction electrodes 44 and 46.

In the radial direction, a distance R3 of the outer peripheral side end portion 91b of the first electrode member 91 from the center axis CL1 is equal to or greater than the distance R1 of the outer peripheral side end portion 44b of the first extraction electrode 44 from the center axis CL1. In the radial direction, a distance R3′ of the outer peripheral side end portion 91b of the first electrode member 91 from the extraction opening 66 (here, the reference line SL3) is equal to or greater than the distance R1′ of the outer peripheral side end portion 44b of the first extraction electrode 44 from the extraction opening 66 (here, the reference line SL3). That is, the outer peripheral side end portion 91b of the first electrode member 91 is disposed at the same position as the outer peripheral side end portion 44b of the first extraction electrode 44 in the radial direction, or is disposed on the outer peripheral side of the end portion 44b. In addition, in the example illustrated in FIG. 4, the outer peripheral side end portion 91b of the first electrode member 91 is disposed on the outer peripheral side of the outer peripheral side end portion 69b of the end wall 69.

In the radial direction, the distance R3 of the outer peripheral side end portion 91b of the first electrode member 91 from the center axis CL1 is equal to or less than the distance R2 of the inner peripheral side end portion 84a of the coil 84 from the center axis CL1. In the radial direction, the distance R3′ of the outer peripheral side end portion 91b of the first electrode member 91 from the extraction opening 66 (here, the reference line SL3) is equal to or less than the distance R2′ of the inner peripheral side end portion 84a of the coil 84 from the extraction opening 66 (here, the reference line SL3). That is, the outer peripheral side end portion 91b of the first electrode member 91 is disposed at the same position as the inner peripheral side end portion 84a of the coil 84 in the radial direction, or is disposed on the inner peripheral side of the end portion 84a.

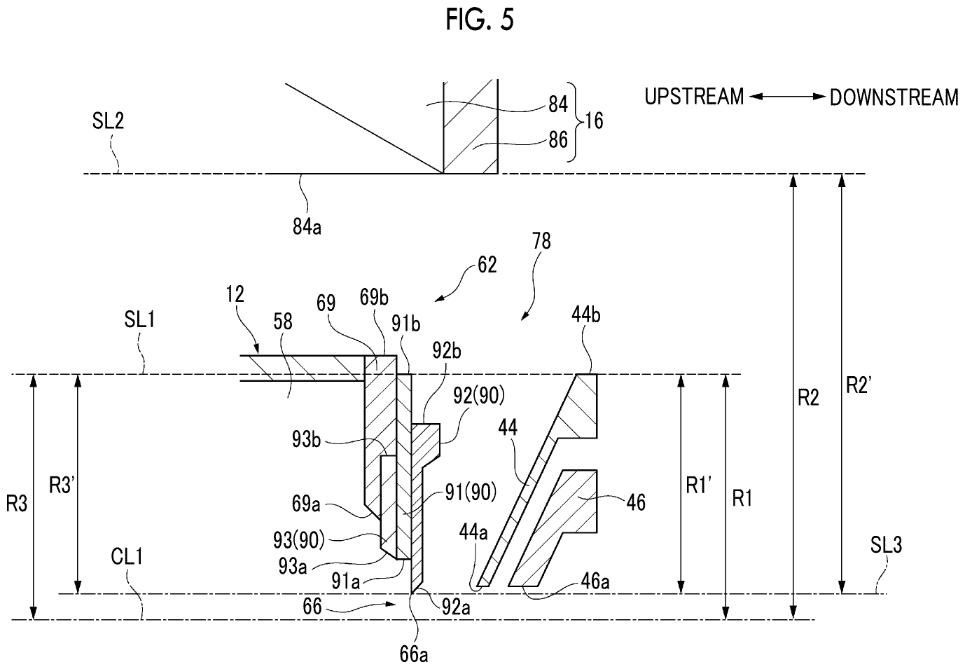

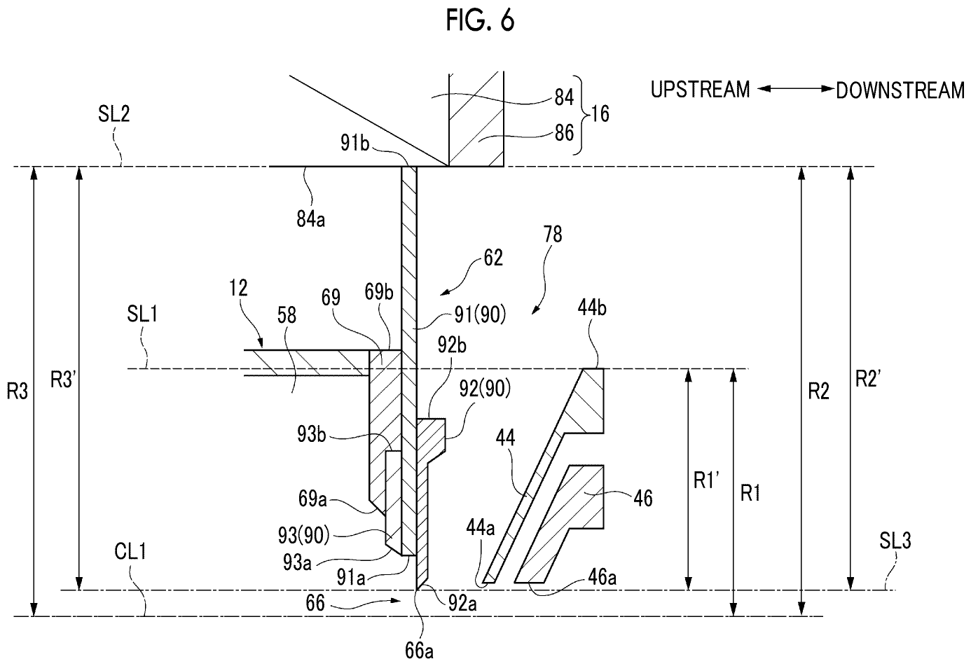

Therefore, the outer peripheral side end portion 91b of the first electrode member 91 may be disposed on the reference line SL1, the reference line SL2, or in a region between the reference line SL1 and the reference line SL2 in the radial direction. FIG. 5 illustrates a state where the outer peripheral side end portion 91b of the first electrode member 91 is disposed at the same position (on the reference line SL1) as the outer peripheral side end portion 44b of the first extraction electrode 44 in the radial direction. FIG. 6 illustrates a state where the outer peripheral side end portion 91b of the first electrode member 91 is disposed at the same position (on the reference line SL2) as the inner peripheral side end portion 84a of the coil 84 in the radial direction. In this state, the first electrode member 91 reaches the magnetic field generator 16.

The second electrode member 92 of the plasma electrode 90 has a substantial disk shape that spreads in the radial direction around the center axis CL1. The second electrode member 92 has a portion protruding to the downstream side in the axial direction in the vicinity of the outer peripheral side end portion 92b, but the shape thereof is not particularly limited. The second electrode member 92 has an inner peripheral side end portion 92a and the outer peripheral side end portion 92b. A positional relationship of the inner peripheral side end portion 92a of the second electrode member 92 can be appropriately changed within a range in which the extraction of the ions is not affected, and the inner peripheral side end portion 92a is disposed such that a required radius can be secured as the extraction opening 66. In addition, the inner peripheral side end portion 92a of the second electrode member 92 may be disposed on the inner peripheral side of the inner peripheral side end portion 91a of the first electrode member 91 so as to be able to protect the first extraction electrode 44 from the discharge. The outer peripheral side end portion 92b of the second electrode member 92 is disposed in a range capable of covering the first electrode member 91 in a range in which the discharge from the first extraction electrode 44 may occur. In the example illustrated in FIG. 4, the outer peripheral side end portion 92b of the second electrode member 92 is disposed on the inner peripheral side of the outer peripheral side end portion of the first extraction electrode 44.

The third electrode member 93 of the plasma electrode 90 has a disk shape that spreads in the radial direction around the center axis CL1. The third electrode member 93 has an inner peripheral side end portion 93a and an outer peripheral side end portion 93b. The positional relationship of the inner peripheral side end portion 93a of the third electrode member 93 can be appropriately changed within a range in which the extraction of the ions is not affected. However, the third electrode member 93 is disposed at a position where the first electrode member 91 is not exposed so that the first electrode member 91 can be protected from the plasma. In addition, the outer peripheral side end portion 93b of the third electrode member 93 is disposed on the outer peripheral side of the inner peripheral side end portion 69a of the end wall 69 so that the first electrode member 91 can be protected from the plasma.

The operation and effects of the ion source 10 and the neutron capture therapy apparatus 100 according to the present embodiment will be described.

In the ion source 10 according to the present embodiment, the plasma electrode 90 has at least the first electrode member 91 and the second electrode member 92, and the first electrode member 91 is a magnetic body. Accordingly, a magnetic field is allowed to escape to the outer peripheral side by the first electrode member 91 which is a magnetic body, so that a leaked magnetic field to between the plasma electrode 90 and the first extraction electrode 44 can be suppressed. In particular, in the radial direction (second direction) perpendicular to the axial direction (first direction) in which the plasma electrode 90 and the first extraction electrode 44 face each other, the distance R3′ of the outer peripheral side end portion 91b of the first electrode member 91 from the extraction opening 66 (here, the reference line SL3) is equal to or greater than the distance R1′ of the outer peripheral side end portion 44b of the first extraction electrode 44 from the extraction opening 66 (here, the reference line SL3). Accordingly, it is possible to suppress the generation of a leaked magnetic field from the outer peripheral side of the first electrode member 91. For example, in a case where the outer diameter of the magnetic body of the first electrode member 91 is smaller than the outer diameter of the first extraction electrode 44, there is a possibility that a leaked magnetic field may be generated from the outer peripheral side end portion 91b of the first electrode member 91 to the space between the electrodes. In the present embodiment, by suppressing the generation of a leaked magnetic field, the generation of discharge between the plasma electrode 90 and the first extraction electrode 44 can be suppressed. In addition, the second electrode member 92 is a member having at least one of a melting point and thermal conductivity higher than those of the magnetic body. During the discharge, electrons run from the extraction electrode 44 toward the plasma electrode 90, causing the plasma electrode 90 to be locally heated at the discharge point. In a case where the heating point is the magnetic body, damage such as melting and cracking of the material occurs due to a momentary temperature rise. In contrast, in the present embodiment, even when the discharge occurs, the first electrode member 91, which is the magnetic body, can be protected by the second electrode member 92. Even when the temperature locally rises due to the discharge, the high melting point material can withstand the temperature rise. In addition, even when the temperature rises, the heat can be quickly diffused as long as a material with high thermal conductivity is provided. From the above, the influence of the discharge between the electrodes can be reduced. In addition, by suppressing the damage to the first electrode member 91, the frequency of failure and the frequency of maintenance can be reduced.

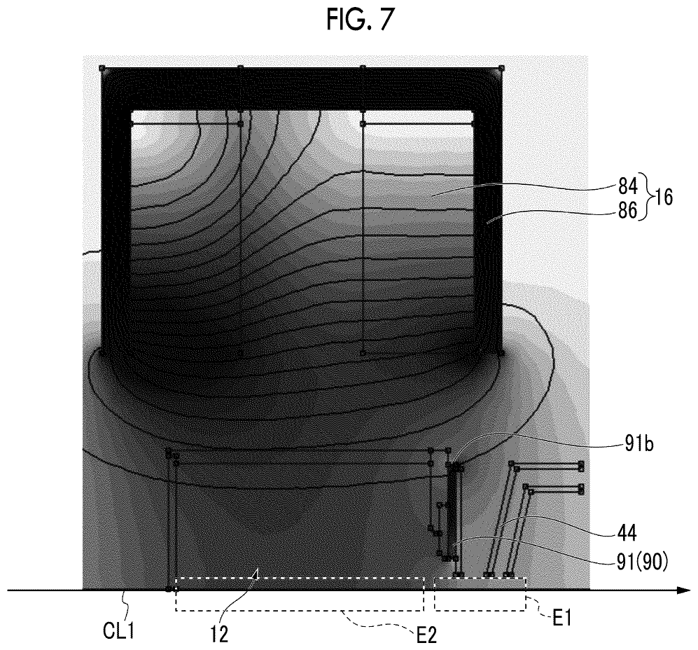

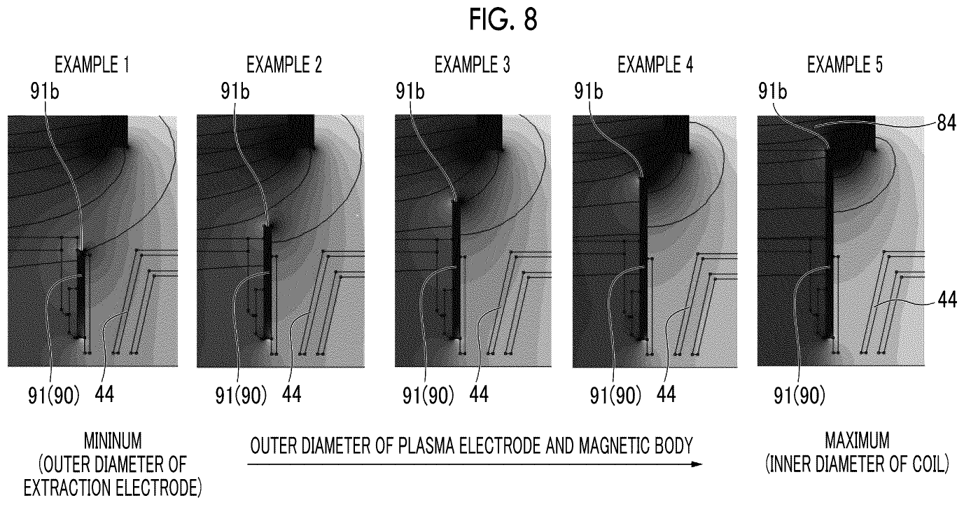

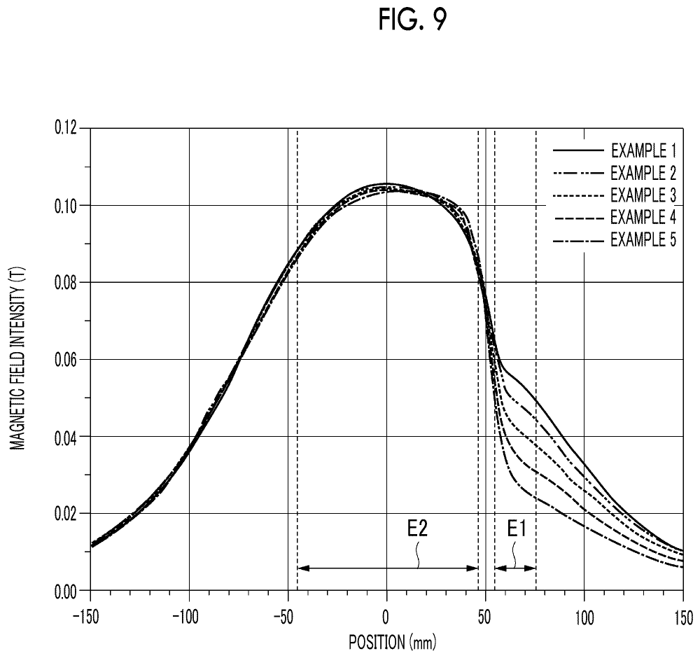

Here, a relationship between the outer diameter of the first electrode member 91 and the effect of suppressing the leaked magnetic field will be described with reference to FIGS. 7 to 9. FIG. 7 is a diagram illustrating results obtained by creating a model of the ion source 10 and simulating a two-dimensional magnetic field calculation system. In FIG. 7, the darker the color, the stronger the magnetic field. In the present simulation, “FEMM” was used as calculation software. As illustrated in FIG. 8, models of Example 1 to Example 5 were prepared. Example 1 is a model in a case where the outer diameter of the first electrode member 91 is substantially the same as the outer diameter of the first extraction electrode 44. Examples 2 to 4 are models in which the outer diameter of the first electrode member 91 is gradually increased. Example 5 is a model in a case where the outer diameter of the first electrode member 91 is the same as the inner diameter of the coil 84. FIG. 9 is a graph illustrating the magnetic field intensity on the center axis CL1 in Example 1 to Example 5. A region of “E1” in the graph indicates a beam extraction region indicated by “E1” in FIG. 7. A region of “E2” in the graph indicates a region of the plasma chamber 12 indicated by “E2” in FIG. 7. As illustrated in FIGS. 9A and 9B, as the outer diameter of the magnetic body increases, the generation of a leaked magnetic field in the beam extraction region can be reduced.

The material of the second electrode member 92 may be selected from tungsten, tantalum, and molybdenum. In this case, the second electrode member 92 can be made to have a high melting point.

The material of the second electrode member 92 may be selected from copper, silver, and aluminum. In this case, the second electrode member 92 can be made to have high thermal conductivity.

The ion source 10 may further include a magnetic field generator 16 provided on the outer peripheral side of the plasma chamber 12 and generating a magnetic field in the axial direction in the plasma chamber 12, and the outer peripheral side end portion 91b of the first electrode member 91 may be disposed at a position separated from the magnetic field generator 16 toward the inner peripheral side. Even in this case, it is possible to suppress the generation of a leaked magnetic field from the outer peripheral side end portion 91b of the first electrode member 91.

The ion source 10 may further include a magnetic field generator 16 provided on the outer peripheral side of the plasma chamber 12 and generating a magnetic field in the axial direction in the plasma chamber 12, and the first electrode member 91 may reach the magnetic field generator 16. In this case, it is possible to further suppress the generation of a leaked magnetic field from the outer peripheral side end portion 91b of the first electrode member 91.

The neutron capture therapy apparatus 100 according to the present embodiment is a neutron ray 100 includes an accelerator 112 that has an ion source 10 for generating ions and that accelerates the ions to emit a particle ray, and an irradiation unit that generates a neutron ray by the particle ray and irradiates an object with the neutron ray, the ion source 10 includes a plasma electrode 90 that is provided in a plasma chamber 12 that generates the ions by plasma, and a first extraction electrode 44 that faces the plasma electrode 90 and extracts the ions from the plasma chamber 12, the plasma electrode 90 has at least a first electrode member 91 and a second electrode member 92, and is formed with an extraction opening 66 through which the ions are extracted, the first electrode member 91 is a magnetic body, and the second electrode member 92 is a member having at least one of a higher melting point and thermal conductivity than those of the magnetic body, and in a radial direction (second direction) perpendicular to an axial direction (first direction) in which the plasma electrode 90 and the first extraction electrode 44 face each other, a distance R3′ of an outer peripheral side end portion 91b of the first electrode member 91 from an extraction opening 66 (here, a reference line SL3) is equal to or greater than a distance R1′ of an outer peripheral side end portion 44b of the first extraction electrode 44 from the extraction opening 66 (here, the reference line SL3).

In the neutron capture therapy apparatus 100 according to the present embodiment, the plasma electrode 90 has at least the first electrode member 91 and the second electrode member 92, and the first electrode member 91 is a magnetic body. Accordingly, a magnetic field is allowed to escape to the outer peripheral side by the first electrode member 91 which is a magnetic body, so that a leaked magnetic field to between the plasma electrode 90 and the first extraction electrode 44 can be suppressed. In particular, in the radial direction (second direction) perpendicular to the axial direction (first direction) in which the plasma electrode 90 and the first extraction electrode 44 face each other, the distance R3′ of the outer peripheral side end portion 91b of the first electrode member 91 from the extraction opening 66 (here, the reference line SL3) is equal to or greater than the distance R1′ of the outer peripheral side end portion 44b of the first extraction electrode 44 from the extraction opening 66 (here, the reference line SL3). Accordingly, it is possible to suppress the generation of a leaked magnetic field from the outer peripheral side of the first electrode member 91. In this way, by suppressing the generation of the leaked magnetic field, the generation of the discharge between the plasma electrode 90 and the first extraction electrode 44 can be suppressed. In addition, the second electrode member 92 is a member having at least one of a melting point and thermal conductivity higher than those of the magnetic body. Therefore, even when the discharge occurs, the first electrode member 91, which is the magnetic body, can be protected by the second electrode member 92. From the above, the influence of the discharge between the electrodes can be reduced. In addition, by suppressing the damage to the first electrode member 91, it is possible to reduce the frequency of a failure that causes the neutron ray N to be unable to be emitted, and it is possible to reduce the frequency of maintenance.

The present invention is not limited to the above-described embodiment.

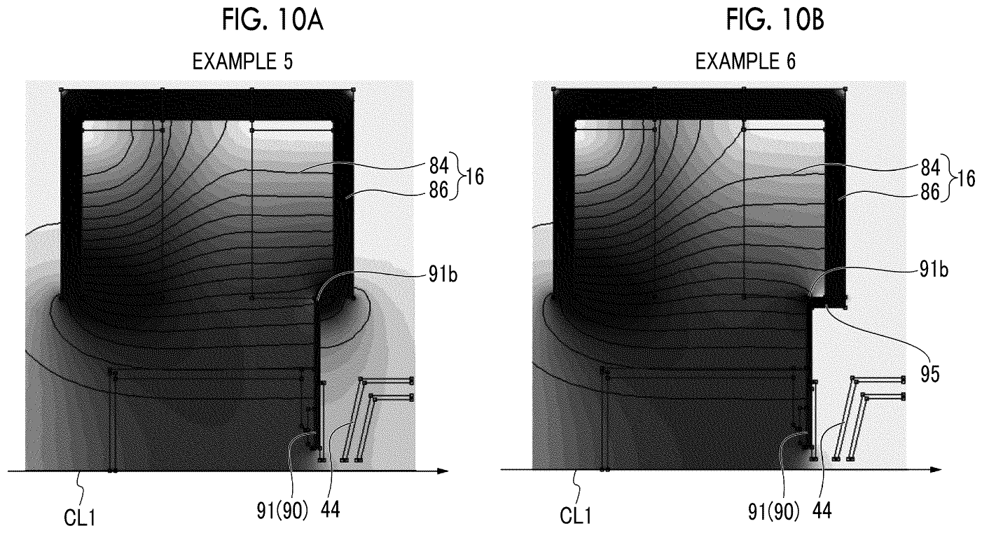

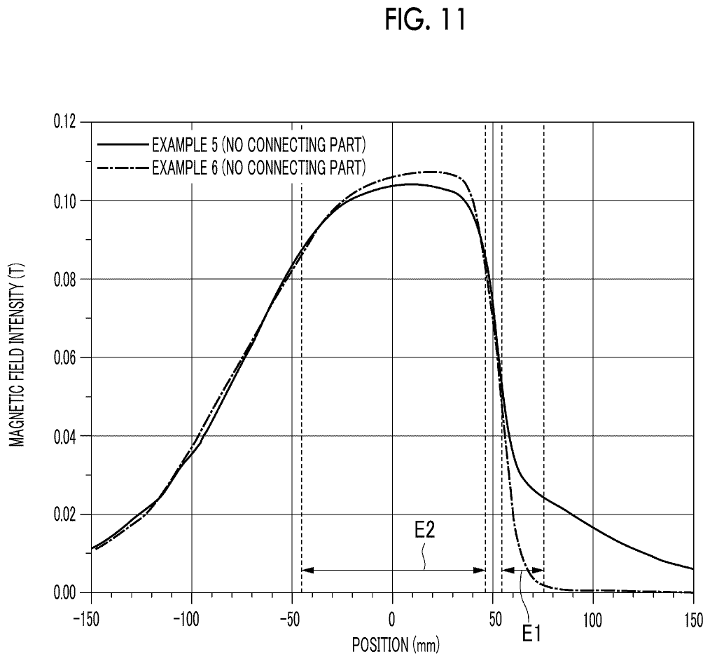

For example, as illustrated in Example 6 of FIG. 10B, the first electrode member 91 may include a connecting part 95 connected to the yoke 86 of the magnetic field generator 16. In Example 6, the first electrode member 91 reaches the coil 84, and the end portion 91b and the yoke 86 are connected to each other by the connecting part 95. In this case, the connecting part 95 allows the first electrode member 91 to be connected to a magnetic circuit with the coil 84, and the generation of a leaked magnetic field can be further suppressed. FIG. 10A illustrates a form in which the first electrode member 91 reaches the coil 84 but does not have the connecting part 95, and corresponds to Example 5 in FIG. 8. As illustrated in FIG. 11, compared to Example 5 that does not have the connecting part 95, Example 6 having the connecting part 95 can significantly reduce the leaked magnetic field in the beam extraction region. In this way, even when the outer diameter of the magnetic body is the same, the degree of reduction of a leaked magnetic field can be further improved depending on the shape. In this way, the shape of the magnetic body is not limited to the disk shape, and n axisymmetric shape in general may be adopted such that a point which protrudes in the axial direction is provided.

In the above-described embodiment, the extraction electrodes 44 and 46 are inclined. However, the extraction electrodes 44 and 46 may be disk-shaped electrodes that spread straight in the radial direction. In a case where the extraction electrodes 44 and 46 have a disk shape, the discharge is likely to occur even on the outer peripheral side. Thus, the second electrode member 92 preferably covers the first electrode member 91 up to a position on the outer peripheral side of the extraction electrode 44.

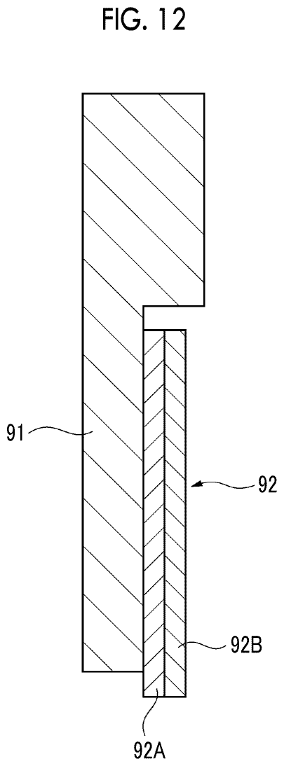

The second electrode member 92 is not limited to the above-described embodiment. For example, as illustrated in FIG. 12, the second electrode member 92 may have a multilayer structure. Here, the second electrode member 92 has a first layer 92A having high thermal conductivity on the upstream side and a second layer 92B having a high melting point on the downstream side. The second layer 92B on the downstream side has a higher temperature during discharge. Thus, by making the second layer 92B have a high melting point, the second electrode member 92 having excellent durability can be obtained.

In the above-described embodiment, the ion source 10 is applied to the accelerator 112 of the neutron capture therapy apparatus 100. However, the use of the ion source 10 is not limited, and the ion source 10 is used, for example, as an ion source for an ion implanter or another particle ray therapy apparatus. The ion source 10 is used as, for example, a monovalent ion source. In addition, the ion source 10 may be used as an ion source for a proton accelerator or as an X-ray source.

The above-described ion source 10 extracts the positive ions. However, whether the generated ions are positive ions or negative ions is not particularly limited.

In addition, the ion source 10 is not limited to the microwave ion source, and can be applied to a general ion source (for example, an ECR ion source) using a magnetic field.

It should be understood that the invention is not limited to the above-described embodiment, but may be modified into various forms on the basis of the spirit of the invention. Additionally, the modifications are included in the scope of the invention.

What is claimed is:

1. An ion source comprising:
a plasma electrode that is provided in a plasma chamber in which ions are generated by a plasma; and
an extraction electrode that faces the plasma electrode and extracts the ions from the plasma chamber,
wherein the plasma electrode includes at least a first electrode member and a second electrode member, and is formed with an extraction opening through which the ions are extracted,
the first electrode member is a magnetic body,
the second electrode member is a member having at least one of a melting point and thermal conductivity higher than those of the magnetic body, and
in a second direction perpendicular to a first direction in which the plasma electrode and the extraction electrode face each other, a distance of an outer peripheral side end portion of the first electrode member from the extraction opening is equal to or greater than a distance of an outer peripheral side end portion of the extraction electrode from the extraction opening.

2. The ion source according to claim 1,
wherein the plasma electrode is an electrode that is provided in the plasma chamber and includes the extraction opening that is open from the plasma chamber in an axial direction.

3. The ion source according to claim 2,
wherein the plasma electrode spreads in a direction vertical to the axial direction toward an outer peripheral side around a center axis.

4. The ion source according to claim 3,
wherein the plasma electrode has an axisymmetric shape with the center axis as a reference.

5. The ion source according to claim 1,
wherein the first electrode member has a disk shape that spreads in a radial direction around a center axis.

6. The ion source according to claim 1,
wherein the second electrode member is provided on a downstream side of the first electrode member in an axial direction.

7. The ion source according to claim 6,
wherein the second electrode member is a non-magnetic body.

8. The ion source according to claim 7,
wherein the second electrode member has a disk shape that spreads in a radial direction around a center axis.

9. The ion source according to claim 8,
wherein the extraction electrode includes a first extraction electrode and a second extraction electrode, and an outer peripheral side end portion of the second electrode member is disposed in a range capable of covering the first electrode member, within a range in which discharge from the first extraction electrode occurs.

10. The ion source according to claim 9,
wherein the outer peripheral side end portion of the second electrode member is disposed on an inner peripheral side of the outer peripheral side end portion of the first extraction electrode.

11. The ion source according to claim 1,
wherein the extraction opening is formed by a through-hole that penetrates an end wall, the first electrode member, the second electrode member, and a third electrode member in an axial direction.

12. The ion source according to claim 11,
wherein the extraction opening is formed at a position facing a vacuum window with a plasma generation space interposed therebetween.

13. The ion source according to claim 1,
wherein a material of the second electrode member is selected from a group consisting of tungsten, tantalum, and molybdenum.

14. The ion source according to claim 1,
wherein a material of the second electrode member is selected from a group consisting of copper, silver, and aluminum.

15. The ion source according to claim 1, further comprising:
a magnetic field generation unit that is provided on an outer peripheral side of the plasma chamber and generates a magnetic field in the first direction in the plasma chamber,
wherein the outer peripheral side end portion of the first electrode member is disposed at a position separated from the magnetic field generation unit toward an inner peripheral side.

16. The ion source according to claim 1, further comprising:
a magnetic field generation unit that is provided on an outer peripheral side of the plasma chamber and generates a magnetic field in the first direction in the plasma chamber,
wherein the first electrode member reaches the magnetic field generation unit.

17. The ion source according to claim 16,
wherein the magnetic field generation unit includes a coil and a yoke, and
the first electrode member includes a connecting part that is connected to the yoke of the magnetic field generation unit.

18. A neutron capture therapy apparatus comprising:
an accelerator that includes an ion source for generating ions, and that accelerates the ions to emit a particle ray; and
an irradiation unit that generates a neutron ray by the particle ray and irradiates an object with the neutron ray,
wherein the ion source includes
a plasma electrode that is provided in a plasma chamber in which ions are generated by a plasma, and
an extraction electrode that faces the plasma electrode and extracts the ions from the plasma chamber,
the plasma electrode includes at least a first electrode member and a second electrode member, and is formed with an extraction opening through which the ions are extracted,
the first electrode member is a magnetic body,
the second electrode member is a member having at least one of a melting point and thermal conductivity higher than those of the magnetic body, and
in a second direction perpendicular to a first direction in which the plasma electrode and the extraction electrode face each other, a distance of an outer peripheral side end portion of the first electrode member from the extraction opening is equal to or greater than a distance of an outer peripheral side end portion of the extraction electrode from the extraction opening.
